(19) United States
(12) Patent Application Publication
(10) Pub. No.: US 2006/0079463 A1
(43) Pub. Date: Apr. 13, 2006

(54) Anticancer compositions comprising methenamine

(76) Inventors: Hong Ji Zhong, San Francisco, CA (US); Zhi-Ying Chen, Foster City, CA (US)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/283,468

(22) Filed: Nov. 18, 2005

Related U.S. Application Data
(63) Continuation-in-part of application No. PCT/US04/16455, filed on May 20, 2004.
(60) Provisional application No. 60/471,966, filed on May 20, 2003.

(51) Int. Cl. A61K 38/04; A61K 31/53; A61K 31/60

(52) U.S. Cl. 514/19; 514/159; 514/246

(57) ABSTRACT

The invention is directed to anti-cancer compositions comprising methenamine or its derivatives or conjugates, and to use of such methenamine containing compositions to treat cancer.

This application is a Continuation-in-Part application of PCT/US2004/016455, filed May 20, 2004, which claims the benefit of U.S. Ser. No. 60/471,966, filed May 20, 2003, the contents of these applications are incorporated by reference into this application.

### FIELDS OF THE INVENTION

The present invention relates to the use of methenamine and methenamine containing compounds for treating cancer.

### BACKGROUND OF THE INVENTION

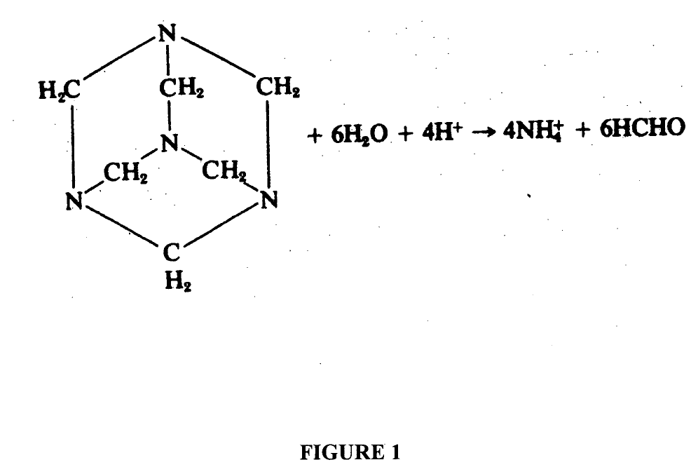

Methenamine (also known as hexamine, hexamethylenetetramine, or urotropin; see FIG. 1 for chemical structure) (“URIN” hereinafter) was introduced into clinical use as a urinary antiseptic as long ago as 1894. Its derivatives include various salt forms such as methenamine mandelate (“MAIN” hereinafter), methnamine hippurate, and methenamine sulfosalicylate, which have been used for urinary tract infections, and can be administered orally. Recently, more effective antibiotics, such as Ampicillin and Tetracyclines, have replaced these drugs for treatment of urinary tract infections.

One methenamine molecule is hydrolyzed to 4 molecules of ammonia and 6 molecules of formaldehyde in an acid medium (see also FIG. 1). Once formed, formaldehyde can denature proteins, causing the death of microorganisms and eukaryotic cells. Formaldehyde is the active form of the methenamine and its derivatives, including methenamine mandeiate, methenamine hippurate, and methenamine sulfosalicylate.

Methenamine is very stable in a pH-neutral medium, and does not liberate formaldehyde in serum and normal tissue (Kucers A, et al.: The Use of Antibiotics: A clinical review of anti bacterial, antifungal and antiviral drugs, Fifth Edition. The Bath Press, Avon. 1997, p. 932-935). Hydrolysis of the methenamine moiety and liberation of formaldehyde occurs only in an acidic medium (see FIG. 1) such as acidified urine. Therefore, the use of this class of drugs has been limited to the treatment of lower urinary tract infections. The hydrolysis rate of methenamine increases with an increased acidity of the medium. To enhance their antibiotic effect in treating urinary infections, additional compounds, such as methionine, ascorbic acid, etc., are used to acidify patients' urine and hence an increased production rate of formaldehyde. The methenamine class of drugs has very low toxicity, and is very safe, and “the usually recommended doses are used for long term therapy” (Kucers A, et al., supra).

A U.S. Public Health Service Cooperative Study compared other drugs with methenamine mandelate and placebo in 249 males over a two-year period, and found that the side effects resulting from the long term use of these agents were negligible (Freeman R B, et al.: Long-term therapy for chronic batceriuria in men. Ann Intern Med. 1975; 83:133; Kda-Kimble M A, et. al.: Applied Therapeutics, Applied Therapeutics, Inc. Vancouver, Wash., 1992, p. 43-12 and 13). “No evidence of bone marrow depression, liver damage or peripheral neuritis has been observed when these drugs have been used in recommended doses” (Gibson G R: A clinical appraisal of methenaimne hippurate in urinary track infections. Med J. Aust. 1:83, 1970). Only a very small percent of patients develop gastrointestinal side-effects such as nausea, vomiting and diarrhea. High doses or prolonged administration may lead to urinary tract irritation due to liberated formaldehyde (Kucers A, et al., supra).

Recently researchers have been trying to exploit the tumor increased acidity to enhance the anticancer effect of acid-labile prodrugs with limited success (Rong Zhou, et al.: Intracellular acidification of human melanoma xenograph by the respiratory inhibition m-iodobenzylguanidine plus hyperglycemia: a 31 P magnetic resonance spectroscopy study. Cancer Research 60:3532-6, 2000; Stubbs M, et al.: Causes and consequences of acidic pH in tumors: a magnetic resonance study. Advance Enzyme Regulation, 39:13-30, 1999). A variety of chemicals and methods have been demonstrated to further lower the pH values of tumors experimentally and clinically (Rong Zhou, et al., supra).

Cancer remains for many patients an incurable disease. It would be a great advantage to medicine to develop drugs to more effectively treat cancer with limited or no side effects to the treatment.

### SUMMARY OF THE INVENTION

This invention includes anti-cancer compositions comprising methenamine or methenamine derivatives including salts, and methenamine conjugates, with a pharmaceutically acceptable carrier appropriate for the mode of the delivery and cancer being treated. The invention also includes methods of treating patients having cancer comprising administering such anti-cancer compositions, and methods of making the compositions proposed. The invention further includes combination of methenamine compounds conbined with other treatments to achieve a synergistic effect.

All the references cited herein are incorporated into this application by reference.

### DETAILED DESCRIPTION OF THE FIGURE

FIG. 1. Methenamine and its hydrolysis in an acid pH shown as the scheme of the molecular structure of a methenamine; in an acidic pH environment, one molecule of methenamine reacts with 6 molecules of water and 4 molecules of hydrogen to generate 6 molecules of formaldehyde and 4 molecules of amonia; (Craig C R, and Stitzel R E; Modern Pharmacology, Second Edition; 1986, p. 652; Little and Brown Company, Boston, Toronto.)

### DETAILED DESCRIPTION OF THE INVENTION

Unless otherwise indicated, all terms are generally consistent with the meaning that the terms have to those skilled in the art of the present invention.

It has been known for many years that tumors of human and animals have an acidic pH (Warburg, O. The metabolism of tumors; Arnold Constable, London (1930)). Recently, by using sophisticated technologies including 31 p Magnetic Resonance Spectroscopy (MRS), it has been further demonstrated that it is the extracellular pH that is acidic (pH 6.5-6.8, Griffiths J R et al.: Why are cancer acidic? A carrier-mediated diffusion model for H+ transport in the interstitial fluid. Norvatis Found Symp 2001; 240:46-62, Discussion 62-7), while the intracellular pH of the cancer cells is near-neutral (Stubbs M, et al., supra), or slightly alkaline (Webb S D, et al.: Modelling tumor acidity and invasion. Novartis Found Symp.; 2001; 240:169-81; discussion 181-5). The pH of many human and animal tumors can be more than 0.2 pH units lower than normal tissue (Stubbs M, et al., supra). Usually, the interstitial fluid pH of many tumors is around pH6.5-6.8. In some cases, especially when necrosis occurs, the acidity of a tumor can be as low as pH 5.2.

Although this invention is not limited to theories of how the drug works, this invention discloses treatment of cancer patients with administration of oral or intravenous methenamine compounds or salts or other derivatives of compositions containing methenamine in the acidic extra-cellular microenvironment of cancer cells, methenamine will degrade to liberate formaldehyde which will kill the cancer cells, by denaturing the proteins and other cellular macromolecules. The theorized method of killing cancer cells with methenamine is essentially by the same mechanism by which the drug works in killing the micro-organisms causing lower urinary tract infections. It is anticipated that methenamine and its salts or other derivatives will have a positive therapeutic anti-cancer effect in the cases where the cancer cells exist in vivo in an acidic microenvironment. The acidic microenvironment around the cancer cells is presumed to be created by the cancer cells themselves, either while they grow or during necrosis of the cancer cells. It is reasoned that although formaldehyde is liberated from parent methenamine compounds in interstitial space, it can diffuse to interact with intracellular macromolecules, such as proteins and nuclear acids, and kill cancer cells.

The anti-cancer therapeutic effect of methenamine, its salts and other derivatives will be enhanced by increasing the acidity of the cancer's extracellular microenvironment. Methenamine as such manipulation of the local environment will result in an increased rate of formaldehyde liberation upon administration of methenamine containing compositions. The acid environment enhancement can be achieved by many means. For example, a synergistic effect can be achieved by co-administration of glucose because cancer cells metabolize by aerobic glycolysis, i.e., the cancer cells produce ATP by conversion of glucose to lactic acid, even when adequate supplies of oxygen are available. The lactate ion and H+ will be effluxed into the extracellular fluid rapidly, and consequently, the extracellular pH of the cancer will be further lowered (Stubbs M, et al., supra) with a resulting increased rate of formaldehyde liberation from methenamine upon administration of methenamine containing compositions. This glucose-resulted tissue acidity enhancement will not occur to normal tissue because glucose there can be completely oxidized to become H2O and CO2.

The glucose-selective acidification of tumor tissue could be further enhanced by co-administration of respiratory inhibitor such as m-Iodpbenzylguanidine (MIBG). It has been shown that administration of a dose of MIBG under hyperglycemic conditions reduced the extracellular pH of human melanoma xenographts in SCID mice by up to 0.59 units, i.e., to pH 6.35 to 6.4. (Zhou R, et al., supra). Enhancement of cancer extracellular acidity can also be achieved by additional compounds which regulate a particular set of genes related to cellular respiration and glucose hydrolysis. Fructose 2, 6-bisphosphate, for example, is a powerful regulator of mammalian glycolysis which acts by stimulating the activity of 6-phosphofructo-1-kinase (PFK-1). The intracellular concentration of fructose 2,6-bisphosphate is in turn controlled by the inducible gene product 6-phosphofructo-2-kinase (PFK2)/fructose-2,6-bisphosphatase, an enzyme over-expressed in many human cancers, including colon, breast, and ovarian cancers (Atsumi T et al.: High expression of inducible 6-phosphofructose-2,6-bisphosphatase (iPFK-2; PFKFB3) in human cancers; Cancer Res 62:5881, 2002). In addition to hypoxia, 6-mercaptopurine, all-trans vitamin A, okadaic acid, and xylulos-5-p, are also the regulators of 6-phosphofructo-2-kinase (PFK2)/fructose-2,6-bisphosphatase (El-Maghrabi M R et al.: 6-phosphofructose-2-kinase/fructose-2,6-bisphosphatase: suiting structure to need, in a family of tissue-specific enzymes; Curr Opin Clin Nutr Metab Care 4:411, 2001). Thus, fructose 2, 6-bisphosphate, and 6-mercaptopurine, all-trans vitamin A, okadaic acid, and xylulos-5-p are the candidates as enhancers of glycolysis rate and acidity of cancer.

It has also been shown that selectively increased acidity of tumor tissue can be achieved by localized ultrasound hyperthermia (Kallinowski F, and Vaupel P,: Factors governing hyperthermia-induced pH changes in Yoshida sarcomas. Int J Hyperthermia 1989; 5(5):641-52). In general then, raising the local temperature at the tumor site can increase the acidity of the tumor. Temperature increase at the tumor site can be accomplished by any means known to do so, including, for example, using a tool such that causes direct heating, microwave heating, or light-based heating at the tumor site. Generally, there are methods known in the art of passing wavelengths of energy through the normal tissue to the cancer tissue where the wavelengths provide selective heating to the tumor tissue, for example by the use of radiation. Heating tumor tissue may be accomplished by other means, including but not limited to chemical means, and any appropriate energy aimed or positioned for release at the tumor site. Methods for raising a local temperature at a tumor site are known in the art. Raising the temperature of the tumor causes an increase in localized acidity, compatible with administration of a methenamine-containing drug that will activate in a slightly acidic environment.

This invention also includes the concept that a synergistic effect may be achieved by using specially designed chemical compositions that are stable in neutral-pH medium but degrade in acidic pH to release acid products, resulting in a further decrease in pH value in the cancer extracellular microenvironment. Thus, the methenmine molecule is exemplary and can be used to design chemicals and pharmaceutical compositions that will act selectively on tumor tissue and result in little or no toxicity to normal cells.

Methenamine and its derivatives have potential as a class of very safe chemotherapeutic drugs for administration to treat malignant cancerous tumors. Furthermore, a variety of new anticancer drugs based on the releasing of the effective element formaldehyde in acidic cancer microenvironment can be synthesized, and different enhancing chemicals, methods and devices can be developed to enhance the anticancer effect of methenamines containing compounds and compositions by selectively increasing tumor acidity during administration of the formaldehyde-releasing chemicals. For example, side chains can be added to methenamine, conjugated moieties, multiple repeat molecules of methenamine, and the like can be designed and tested for effectiveness as anti-cancer agents. Particularly, the chemical design process can proceed with a view to exploiting the acidic environment surrounding cancer cells and a continued stability of the molecule in the presence of the essentially neutral (and slightly basic) pH of normal cells (at about pH 7.4). Accordingly, the use of any appropriate methenamine compound, including, but not limited to, methenamine, methenamine salts, other methenamine derivatives, methenamine conjugates (molecule conjugated to methenamine; methenamine conjugated to other methenamines), and other standard alterations in the methenamine molecule or structure that can yield an active methenamine containing chemical that will release formaldehyde in a slightly acidic environment such as the environment encountered in the extracellular space surrounding cancer cells.

Further, it is expected to use methenamine and methenamine containing compositions for treating all cancers, including malignancies found in all tissue types and organs of the body. For a comprehensive list of all cancers known to humans, consult standard medical texts on cancer and the like.

The invention also includes a combination of methenamine with other treatments to produce a synergistic anticancer effect. Other treatments can be chemicals, such as cyclophospharamide (CTX), 5-FU, antibody based therapy, radiotherapy, surgery, and gene therapy, etc. . . .

Other compounds can be designed according to the working principle of methenamine compounds, i.e., to be stable, inert, and non-toxic in any microenvironment with neutral pH, but to be unstable and capable of generating or releasing active molecule(s) and attacking the cellular macromolecules in microenvironment with acidic pH such as cancer interstitial fluid.

Methenamine, also called hexamethylenetetramine, can be synthesized essentially as described in U.S. Pat. No. 2,762,799 and U.S. Pat. No. 2,762,780. Methenamine and its salts are also readily available from pharmacists and drug companies by prescription for treatment of urinary tract infections. Some of the methenamine salt derivatives include, e.g. methenamine mandelate, methnamine hippurate, and methenamine sulfosalicylate. Other methenamine containing compounds can be synthesized by standard chemical techniques known in the art of synthetic chemistry. For administration to animals and humans, the methenamine containing compounds are further combined with pharmaceutically acceptable carriers appropriate for the mode of admininstration or for the drug synthesized (or both). For example, intravenous admininstration requires an IV formulation; and oral admininistration requires formulation of the drug into a tablet or other format for ingestion by mouth. Other non-parenteral administrations may require different formulations, for example cream formulations for topical administration, (e.g. perhaps also with an acidying agent to act. upon exposure to skin or air), or suppository formulations for administering the active agent methenamine containing compound via a cavity, or also by example an intratumoral formulation for administration into a tumor.

Administration of methenamine mandelate can be by any mode deemed optimal for the type of cancer being treated, including but not limited to oral administration and parenteral administration. Parenteral administration can comprise, for example, intravenous, intramuscular, intra-tumoral, intra-organ and other administrations, not limited to those listed here. Needles, catheters, pumps, time-release units, oral formulations and the like may be exploited to achieve an effective delivery of the drug.

The dosage of the drug may vary depending on such patient-based parameters as the size and weight of the patient, the extent of the cancerous involvement, the tolerance of the patient to the drug, and the type and aggressiveness of the cancer, and mode of administration of the drug. The same variables may dictate the timing of administration, for example, whether a bolus of the drug is given daily in the morning, or daily throughout the day, weekly, morning and night, and the like. The duration of the treatment period likewise depends on similar variables, including also the responsiveness of the patient's cancer to the treatment. For extensive cancers, for example, a dosage continued daily for up to 5 years might be optimal. For others, several months may suffice. Still for others administration might be best achieved by several months to a year of continued administration, followed by a resting period of, for example, a month or several months, followed in turn by a resumption of the administration of the drug. In any event, recurrence of the cancer would also dictate repeated administration of the drug, perhaps at a higher dose or with greater frequency or for a longer duration of treatment period. Non-toxic dosages are confirmed at the dosage amount for treating urinary tract infections.

In all cases, the individuals being treated are first diagnosed with cancer by standard methods. Administration of the methenamine or its derivatives is then begun at an appropriate dosage for the patient's size, condition and type of cancer, using a mode of administration (e.g. oral or parenteral) previously determined to be optimal for that type of cancer. Subsequent periodic assessment of the patient's condition is made using standard techniques for detecting and prognosing cancer, e.g. CT scan, marker evaluations, and the like. Once the patient is cancer free, the administration can continue for sometime in order to make sure that all the errant cancer cells have been eliminated. Follow-up care for the patient can occur at regular monthly or bimonthly or quarterly intervals, for example for the first year, and thereafter annually, for example. In all cases care of the patients is adjusted by the information developed for the particular patient at issue, including their starting condition and responsiveness to the drug.

Exemplary intravenous administrations can include the following: patients are administered three consecutive daily doses of 20 grams of urotropin each mixed with 500 ml of 10% glucose and infused intravenously. There is a two-day interval before the second course is started. Each patient receives 3 courses of urotropin treatment.

Exemplary administration for patients who receive the drug orally, for example the salt derivative of methenamine, methenamine mandelate, can be 2 grams of the oral drug twice a day, a same dose level recommended for urinary tract infections. Patients can recieve three doses daily for two weeks, and then return to a two doses daily. The duration of the oral administration can exceed 3 years if necessary.

As it can easily be appreciated in the field, other routes of administration may be used.

### EXAMPLES

The invention is now illustrated by a number of examples.

### Example 1

### Intravenous Admininistration

Patients are selected for administration of intravenous urotropin (methenamine for intravenous administration). The patient's cancer is identified using standard methods, e.g. CT scans and blood marker analysis. Three daily doses of urotropin, 20 grams each in 500 ml of 10% glucose solution, are infused intravenously followed by a 2-day interval. Three courses are administered. The patient is observed for side effects and effectiveness of the treatment. The amount per dose can be adjusted. It usually is 20 grams per day for a 60 kg adult, but can be decreased to 0.1 gram or increased to 200 grams or even more. The number of doses per day can also be modified: it can be once daily, or 2 to 3 doses daily (or even more), or it can be given continuously. The time between two methenamine administrations can be from one day to 10 days or even longer. Generally, the regimen of methenaime administration is adjustable.

### Example 2

### Oral Administration

Patients are selected for oral administration of methenamine salts, e.g. methenamine mandelate. The patient's cancer is identified using standard methods, e.g. CT scans and blood marker analysis. The patient is administered 2.0 grams of methenamine (urised, 500 mg/tablet, ×4 tablets/dose) orally twice per day. The methenaime treatment protocol is adjustable, including amount per dose, number of doses per day, and the interval between two methenamine treatments, as stated in Example 1, is adjustable. Follow-up marker and CT scan data is collected after two months. The patient is directed to remain on the oral administration protocol for several more months. The dosage of the drug can be reduced upon positive response to the cancer, after the patient is determined to be cancer-free by standard testing methods.

### Example 3

### Animal Study—Intravenous Administration

Urotropin (methenamine for injection) is tested in several mouse tumor models. For testing by intravenous administration, three groups of mice, 10 mice in each group are used. After inoculation of each mouse with tumor cells, two groups in the IV study are injected intrapeitoneally with urotropin 2.8 grams per kilogram in the first group and 3.6 grams per kilogram in the second group, twice daily for 3 days. An oral dose of 10% glucose, 20 ml per kilogram of mouse is given 1.5 hours before each urotropin dose. A third control group is given glucose only. The mice are observed for decrease in tumor size and metastasis among other standard parameters of recovery or response in tumor containing mice.

Several mouse models are tested in this fashion, selecting various types of cancer for testing.

### Example 4

### Animal Study—Oral Administration

Oral methenamine (methenamine mandelate) is tested in several mouse tumor models. For oral testing, three groups of mice, 10 mice in each group are used. After inoculation of each mouse with tumor cells, two groups in the oral study are given a dose or methenamine mandelate analogous to the dosage given adults who are treated for urinary tract infections, taking into consideration the weight of the mice. One of those two groups is also administered oral glucose 1.5 hours before methenamine mandelate administration. A third group is administered glucose alone. The methenamine mandelate oral administration is twice daily for 1 week, after which tumor size and the presence of metastasis is observed.

Mice will also be tested for responsiveness to increased or reduced quantities of the drug, and for responsiveness in concert with acidifying agents taken with the methenamine mandelate. In all cases, further observation for effects on the cancerous tumors are observed after an appropriate time period.

### EXPERIMENTAL

### I. Experimant Prorotocol

A. Chemicals and Animals: URIN and MAIN were purchased from Sigma-Aldrich (St Louis, Mo., USA). C57BL/6 mice were purchased from Guangzhou Military Medical University (Guangzhou, China), and Kunming mice from Sun-Yet Sen University (Guangzhou, China).

B. Animal Tumor Models: The anticancer efficacy of URIN and MAIN was determined using 3 xenograft tumor models.

(1) Kunming mouse Sarcoma S-180 model: Mouse Sarcoma S-180 ascites tumor cells, 1×10E6 cells resuspended in 0.2 ml saline per mouse, were injected subcutaneously to the right axillary space of 5- to 6-week old Kunming mice.

(2) Kunming mouse Hepatoma H22 model: Mouse Hepatoma H22 ascites tumor cells, 1×10E6 cells in 0.2 ml saline per mouse, were injected subcutaneously to the right axillary space of 5- to 6-week old Kunming mice.

(3) C57BL/6 mouse melanoma B16 model: Mouse melanoma B16 cells, 1×10E7 cells in 0.2 ml of saline per mouse, were injected subcutaneously into right axillary space of 6 to 8 week old C57BL/6 mice. The mice were randomly divided into groups after tumor cell inoculation.

C. Drug Administration: URIN was dissolved in distilled water and the pH of the solution was adjusted to 7.4 by adding the appropriate amount of MAIN. MAIN solution was made by adding the appropriate amount of the drug to a combination of distilled water and 10N sodium hydroxide with a predetermined ratio ensuring a pH 7.4 after the drug was dissolved completely. The amount of either tested drug for one kg mouse was prepared as 10 ml solution, and 10 micro-liters per gram of mouse were injected intraperitoneally, once or twice daily, for 10 consecutive days for the Kunming mice with Sarcoma S-180 and Hepatoma H22, and for 15 consecutive days for C57BL/6 mice with melanoma B16. 50% glucose, 30 ml/kg, was given by gavage to selected groups of mice receiving the tested drug 60 to 90 minutes before each injection. One group of mice received saline only while another group of mice received the glucose treatment without the tested drug; these groups served as the saline-only control and the glucose-only control, respectively. A third group of mice received intraperitoneal injection of cyclophosphamide (CTX), or 5-Fluorouracil (5FU), 25 to 50 mg/kg body weight, was included in some experiments as positive control. At the end of the experiment, mouse body weight was taken, and the Sarcoma S-180, or Hepatoma H22 tumors were dissected and weighted, while the tumor size of melanoma B16 was measured using a caliper. Melanoma B16 tumor volume was calculated using the following equation: Tumor volume=S*S*L*0.5, where S is the short diameter, and L stands for long diameter of the tumor according to Beck M T et al. (Cancer Research 63:3598, 2003).

### II. Results and Discussion

A. URIN treatment significantly inhibited tumor growth: URIN treatment (4480 mg/kg, i.p. twice daily) significantly reduced the growth of mouse Sarcoma S-180 tumor as compared to mice receiving saline injection (tumor growth inhibition rate 21.9%, p<0.05, Table 1.2). The anticancer effect of URIN was reproducible in the mouse Hepatoma H22 model. A same URIN treatment resulted in significant growth inhibition of the hepatoma (Inhibition rate 41.2%, p<0.001. Table 2.2).

TABLE 1.1
Effect of URIN Treatment on Body Weight of Mice Bearing Sarcoma S-180

| | Treatment | | | No. of mice | | Body weight (Mean ± SD, g) | | |
|---|---|---|---|---|---|---|---|---|
| Group | Drug | Mg/kg | Doses/day | Start | End | Start | End | p value |
| 1 | Saline | 200 | 2 | 10 | 10 | 21.9 ± 1.1 | 28.1 ± 2.3 | |
| | | (μl/each) | | | | | | |
| 2 | CTX | 30 | 1 | 10 | 10 | 21.9 ± 1.1 | 23.5 ± 1.7 | <0.05* |
| 3 | URIN | 4480 | 2 | 10 | 10 | 21.9 ± 1.1 | 26.6 ± 2.1 | |

*As compared to group 1.

TABLE 1.2
Effect of URIN treatment on In Vivo growth of mouse sarcoma S-180

| | | Tumor | | |
|---|---|---|---|---|
| Group | Treatment | Size (Mean ± SD, g) | Inhibition (%) | P value |
| 1 | Saline | 1.37 ± 0.31 | — | — |
| 2 | CTX | 0.53 ± 0.14 | 61.3 | <0.001* |
| 3 | URIN | 1.07 ± 0.31 | 21.9 | <0.05* |

*As compared to group 1.

TABLE 2.1
Effect of URIN Treatment on Body Weight Growth of Mice Bearing Hepatoma H22

| | Treatment | | | No. of mice | | Body weight (Mean ± SD, g) | | |
|---|---|---|---|---|---|---|---|---|
| Group | Drug | Mg/kg | Doses/day | Start | End | Start | End | p value |
| 1 | Saline | 200 | 2 | 10 | 10 | 24.6 ± 1.0 | 35.6 ± 2.3 | |
| | | (μl/each) | | | | | | |
| 2 | CTX | 25 | 1 | 10 | 10 | 24.6 ± 1.0 | 32.6 ± 2.5 | <0.05* |
| 3 | URIN | 4480 | 2 | 10 | 10 | 24.6 ± 1.0 | 33.5 ± 3.2 | |

*As compared to group 1.

TABLE 2.2
Effect of URIN Treatment on In Vivo Growth of Mouse Hepatoma H22

| | | Tumor | | |
|---|---|---|---|---|
| Group | Treatment | Size (Mean ± SD, g) | Inhibition (%) | P value |
| 1 | Saline | 2.33 ± 0.46 | — | — |
| 2 | CTX | 1.21 ± 0.37 | 48.1 | <0.001* |
| 3 | URIN | 1.37 ± 0.39 | 41.2 | <0.001* |

*As compared to group 1.

B. Glucose enhanced URIN anticancer effect: When glucose was given to the mice before URIN injection, the growth of Sarcoma S-180 tumor was further inhibited (inhibition rate 36.2%, p<0.05, Table 3.2). Glucose synergistic anticancer effect was also reproducible in C57BL/6 melanoma B16 model, even though URIN was given only once daily. With pre-treatment of glucose before each URIN injection, significant reduction in tumor burden was seen in the high dose group (inhibition rate 32.1%, p<0.005, Table 4.2).

TABLE 3.1
Effect of URIN Treatment on Body Weight of Mice Bearing Sarcoma S-180

| | Treatment | | | No. of mice | | Body weight (Mean ± SD, g) | | |
|---|---|---|---|---|---|---|---|---|
| Group | Drug | Mg/kg | Doses/day | Start | End | Start | End | p value |
| 1 | Saline | 200 | 2 | 10 | 10 | 21.2 ± 1.0 | 32.6 ± 2.8 | |
| | | (μl/each) | | | | | | |
| 2 | CTX | 30 | 1 | 10 | 10 | 21.4 ± 1.0 | 23.0 ± 2.9 | <0.001*** |
| 3 | Glucose* | 30 | 2 | 10 | 10 | 21.2 ± 1.0 | 32.6 ± 2.7 | |
| | | (ml/kg) | | | | | | |
| 4 | URIN L** | 1680 | 2 | 10 | 10 | 21.2 ± 1.0 | 34.4 ± 2.7 | |
| 5 | URIN M** | 2800 | 2 | 10 | 10 | 21.2 ± 1.0 | 32.0 ± 2.5 | |
| 6 | URIN H** | 4480 | 2 | 10 | 10 | 21.2 ± 1.0 | 28.4 ± 1.8 | <0.001*** |

*Glucose was prepared as 50% solution in dH2O and given by gavage.
**50% gluclose was given to mice by gavage 60 to 90 minutes before each URIN injection.
***As compared to group 1.

TABLE 3.2
Effect of URIN treatment on in vivo growth of mouse Sarcoma S-180

| | | Tumor | | |
|---|---|---|---|---|
| | Treatment | Weight | | |
| Group | Drug | (Mean ± SD, g) | Inhibition Rate (%) | p value |
| 1 | Saline | 1.96 ± 0.60 | NA | |
| 2 | CTX | 0.53 ± 0.22 | 73.0 | <0.001* |
| 3 | Glucose | 1.61 ± 0.62 | 17.8 | |
| 4 | URIN L | 1.54 ± 0.56 | 21.4 | |
| 5 | URIN M | 1.57 ± 0.50 | 19.9 | |
| 6 | URIN H | 1.25 ± 0.41 | 36.2 | <0.005* |

*As compared to group 1.

TABLE 4.1
Effect of URIN Treatment on Body Weight of Mouse with B16 Melanoma

| | Treatment | | | No. of mice | | Body weight (Mean ± SD, g) | | |
|---|---|---|---|---|---|---|---|---|
| Group | Drug | Mg/kg | Doses/day | Start | End | Start | End | p value |
| 1 | Saline | 200 | 1 | 12 | 12 | 20.4 ± 1.6 | 24.2 ± 2.2 | |
| | | (μl/each) | | | | | | |
| 2 | CTX | 50 | 1 | 12 | 12 | 20.3 ± 1.5 | 18.6 ± 1.6 | <0.01*** |
| 3 | Glucose* | 30 | 1 | 12 | 12 | 20.2 ± 1.8 | 23.5 ± 2.1 | |
| | | (ml/kg) | | | | | | |
| 4 | URIN | 1680 | 1 | 11 | 11 | 20.4 ± 1.6 | 23.7 ± 1.7 | |
| | L** | | | | | | | |
| 5 | URIN | 2800 | 1 | 11 | 11 | 20.0 ± 1.6 | 23.0 ± 1.4 | |
| | M** | | | | | | | |
| 6 | URIN | 4480 | 1 | 10 | 10 | 20.7 ± 1.4 | 22.3 ± 1.4 | |
| | H** | | | | | | | |

*Glucose was prepared as 50% solution in dH2O and given by gavage.
**50% gluclose was given to mice by gavage 60 to 90 minutes before each URIN injection.
***As compared to group 1.

TABLE 4.2
Effect of URIN treatment on mouse melanoma B16 growth

| | | Tumor | | |
|---|---|---|---|---|
| | Treatment | Size | | |
| Group | Drug | (Mean ± SD, mm3) | Inhibition (%) | P value |
| 1 | Saline | 4055 ± 1595 | | |
| 2 | CTX | 118 ± 100 | 97 | <0.001* |
| 3 | Glucose | 4744 ± 1570 | (−17.0%)** | |
| 4 | URIN L | 3376 ± 1419 | 16.7 | |
| 5 | URIN M | 4241 ± 1834 | (−14.6%) | |
| 6 | URIN H | 2755 ± 1098 | 32.1 | <0.05* |

*As compared to group 1.
**Value in bracket was the increased rate as compared to group 1.

C. The salt form of URIN (MAIN) also significantly inhibited tumor growth: MAIN is the mandelate salt of URIN, and is given orally in clinic. Demonstration of its efficacy will prove its potential use by oral administration route. Significant reduction in Sarcoma S-180 tumor burden was seen in the group of mice receiving high dose of MAIN (inhibition rate 45.9%, p<0.01, Table 5.2). MAIN anticancer effect was reproducible in mouse Hepatoma H22 model, where a 51.4% inhibition rate (p<0.01, Table 6.2) was demonstrated.

TABLE 5.1
Effect of MAIN Treatment on Mouse Body Weight Growth of Mice Bearing S-180

| | Treatment | | | No. ofmice | | Body weight (Mean ± SD, g) | | |
|---|---|---|---|---|---|---|---|---|
| Group | Drug | Mg/kg | Doses/day | Start | End | Start | End | P value |
| 1 | Glucose* | 30 | 2 | 1 | 9 | 31.9 ± 2.8 | 21.3 ± 1.1 | |
| | | (ml/kg) | | | | | | |
| 2 | MAIN.L** | 1485 | 2 | 2 | 10 | 31.3 ± 3.0 | 21.3 ± 1.2 | <0.05*** |
| 3 | MAIN.M** | 2320 | 2 | 3 | 8 | 29.3 ± 1.6 | 21.5 ± 1.2 | <0.001*** |
| 4 | MAIN.H** | 3714 | 2 | 4 | 10 | 29.2 ± 2.6 | 21.5 ± 1.2 | <0.001*** |

*Glucose was prepared as 50% solution in dH2O and given by gavage.
**50% gluclose was given to mice by gavage 60 to 90 minutes before each MAIN injection.
***As compared to group 1.

TABLE 5.2
Effect of MAIN treatment on in vivo growth mouse Sarcoma S-180

| | | Tumor | | |
|---|---|---|---|---|
| Group | Treatment | Size (Mean ± SD, g) | Inhibition (%) | P value |
| 1 | Glucose | 2.31 ± 0.96 | NA | |
| 2 | MAIN L | 2.14 ± 0.60 | 7.4 | |
| 3 | MAIN M | 1.85 ± 0.62 | 19.9 | |
| 3 | MAIN H | 1.25 ± 0.41 | 45.9 | <0.05* |

*As compared to group 1.

TABLE 6.1
Effect of MAIN administration on body weight of mouse inoculated with Hepatoma H22

| | Treatment | | | No. of mice | | Body weight (Mean ± SD, g) | | |
|---|---|---|---|---|---|---|---|---|
| Group | Drug | Mg/kg | Doses/day | Start | End | Start | End | p value |
| 1 | Saline | 200 | 2 | 10 | 10 | 21.3 ± 2.2 | 35.6 ± 2.2 | |
| | | (μl/each) | | | | | | |
| 2 | 5FU | 25 | 1 | 10 | 9**** | 21.0 ± 1.3 | 27.0 ± 3.0 | <0.001*** |
| 3 | Glucose* | 30 | 2 | 10 | 10 | 20.8 ± 1.0 | 32.0 ± 2.0 | <0.005*** |
| | | (ml/kg) | | | | | | |
| 4 | MAIN L** | 1500 | 2 | 10 | 9**** | 21.0 ± 1.0 | 28.9 ± 1.0 | <0.001*** |
| 5 | MAIN | 2500 | 2 | 10 | 10 | 20.8 ± 1.0 | 29.3 ± 1.2 | <0.001*** |
| | M** | | | | | | | |
| 6 | MAIN | 3750 | 2 | 10 | 8**** | 21.0 ± 1.1 | 28.5 ± 1.5 | <0.001*** |
| | H** | | | | | | | |

*Glucose was prepared as 50% solution in dH2O and given by gavage.
**50% glucose was given to mice, as in Glucose group, 60 to 90 minutes before each MAIN injection.
***As compared to group 1.
****One or two mice died of accident.

TABLE 6.2
Effect of MAIN treatment on in vivo growth of mouse hepatoma H22

| | | | Compared to | | Compared to | |
|---|---|---|---|---|---|---|
| | | Tumor size | Saline group | | Glucose group | |
| Group | Treatment | Mean ± SD, g | Inhibition (%) | P value | Inhibition (%) | P value |
| 1 | Saline | 2.12 ± 0.77 | NA | | | |
| 2 | 5FU | 0.48 ± 0.25 | 77.4 | <0.01* | 71.4 | P < 0.001** |
| 3 | Glucose | 1.68 ± 0.73 | 20.0 | | | |
| 4 | MAIN L | 1.58 ± 0.42 | 25.5 | | 6.0 | |
| 5 | MAIN M | 1.57 ± 0.74 | 25.5 | | 6.5 | |
| 6 | MAIN H | 1.03 ± 0.66 | 51.4 | <0.01* | 38.7 | <0.01** |

*As compared to Saline group.
**As compared to Glucose group.

D. Administration of methenamine drugs resulted in only slight toxicity. During the course of URIN and MAIN administration, all mice looked healthy. No mouse died of URIN toxicity, though there were some mice which died by accident during the experiment. In all four experiments using URIN, either one or two doses daily, no significant animal growth retardation was seen (Table 1.1, 2.1, 3.1, and 4.1). However, significant animal growth retardation was observed in all two studies using the salt form MAIN, even in the mice receiving the low dose level (Table 5.1, and 6.1).

### III. Conclusion

1. Administration of URIN significantly inhibited In Vivo growth of mouse tumors Sarcoma S-180, Hepatoma H22, and B16 Melanoma.

2. URIN anticancer effect could be enhanced by pretreatment of glucose.

3. Mandelate salt form of URIN can also significantly reduce tumor growth.

4. URIN treatment showed no significant toxicity. MAIN administration demonstrated a slight toxicity as indicated by significant animal growth retardation. Accordingly, it was the mandelatic acid, not the urotropin moiety that was toxic to the animal.

5. Our animal experimental data suggest that methenamine compounds will be effective as human anticancer drugs.

### REFERENCES

Kucers A, et al.: Te Use of Antibiotics: A clinical review of anti bacterial, antifungal and antiviral drugs. Fifth Edition. The Bath Press, Avon. 1997, p. 932-935.

Freeman R B, et al.: Long-term therapy for chronic batceriuria in men. Ann Intern Med. 1975; 83:133; Kda-Kimble M A, et. al.: Applied Therapeutics. Applied Therapeutics, Inc. Vancouver, Wash. 1992, p. 43-12 to -13.

Kda-Kimble M A, et. al.: Applied Therapeutics. Applied Therapeutics, Inc. Vancouver, Wash. 1992, p. 43-12 to -13.

Gibson G R: A clinical appraisal of methenaimne hippurate in urinary track infections. Med. J. Aust. 1:83, 1970.

Rong Zhou, et al.: Intracellular acidification of human melanoma xenograph by the respiratory inhibition m-iodobenzylguanidine plus hyperglycemia: a 31 P magnetic resonance spectroscopy study. Cancer Res. 60:3532-6, 2000.

Stubbs M, et al.: Causes and consequences of acidic pH in tumors: a magnetic resonance study. Advan. Enzyme Regul. 39:13-30, 1999.

Craig C R, and Stitzel R E. Modern Pharmacology, Second Edition. 1986, p. 652. Little and Brown Company, Boston, Toronto.

Warburg, O.: The metabolism of tumors. Arnold Constable, London (1930).

Griffiths J R et al.: Why are cancer acidic? A carrier-mediated diffusion model for H+ transport in the interstitial fluid. Norvatis Found. Symp. 2001; 240:46-62, Discussion 62-7.

Webb S D, et al.: Modelling tumor acidity and invasion. Novartis Found Symp. 2001; 240:169-81; discussion 181-5).

Atsumi T et al.: High expression of inducible 6-phosphofructose-2,6-bisphosphatase (iPFK-2; PFKFB3) in human cancers. Cancer Res. 62:5881, 2002.

El-Maghrabi M R et al.: 6-phosphofructose-2-kinase/fructose-2,6-bisphosphatase: suiting structure to need, in a family of tissue-specific enzymes. Curr. Opin. Clin. Nutr. Metab. Care 4:411, 2001.

Kallinowski F. and Vaupel P.: Factors governing hyperthermia-induced pH changes in Yoshida sarcomas. Int. J. Hyperthermia 1989; 5(5):641-52).

1. An anti-cancer composition comprising effective amount of methenamine or its derivative and a pharmaceutically acceptable carrier.

2. The anti-cancer composition as in claim 1, wherein methenamine or its derivative is formulated with a pharmaceutically acceptable carrier.

3. The composition of claim 1 for administration by a mode selected from the group consisting of oral, parenteral, and non-parenteral modes of delivery.

4. The anti-cancer composition as in claim 1, wherein said composition comprises methenamine derivatives selected from the group consisting of methenamine mandelate, methenamine hippurate, and methenamine sulfosalicylate.

5. The anti-cancer composition as in claim 1, wherein the methenamine composition comprises one or more methenamine units linked to another molecule.

6. A method of treating a patient having cancer comprising administering to the patient an anti-cancer composition comprising methenamine and a pharmaceutically acceptable carrier.

7. A method of treating a patient having cancer as in claim 6, wherein administering comprises using a mode of administration selected from the group consisting of oral, parenteral and non-parenteral delivery.

8. A method as in claim 6, wherein administering comprises using a mode selected from the group consisting of oral, intramuscular, rectal, intravenous, intra-tumor, and intracelially delivery.

9. A method as in claim 6, further comprising the step of administering to the patient an agent that lowers the pH of an extracellular microenvironment at cancer sites in the patient's body.

10. A method as in claim 9, wherein the step of administering a pH-lowering agent comprises administering an agent selected from the group consisting of butyric acid, glucose, lactic acid, ascorbic acid, and a respiratory inhibitor.

11. A method as in claim 10, wherein the pH lowering step comprises administering a respiratory inhibitor or glucose hydrolysis enhancer and the respiratory inhibitor or glucose hydrolysis enhancer is selected from the group consisting of diphosphopyridine nucleotide, m-Iodobenzylguanidine, or other chemicals.

12. A method as in claim 6, further comprising a step of increasing a local temperature in a region of the body comprising said cancer using a tool.

13. A method as in claim 12, wherein said increasing temperature step comprises using a mode selected from the group consisting of direct heating, microwave heating, light-based heating, and heating of other physics means.

14. A method as in claim 6, further comprising using a tool for decreasing a local pH value in a region of the body containing cancer.

15. A method as in claim 14, wherein using a tool to decrease a local pH comprises using a tool that causes ischemia in a region of the body comprising said cancer.

16. A method of making a methenamine containing compound for treating a cancer patient comprising providing a compound comprising methenamine and adding a pharmaceutically acceptable carrier appropriate for an administration route of said compound.

17. A method as in claim 16, wherein said methenamine containing compound comprises a compound selected from the group consisting of methenamine, urotropin, a methenamine salt, a methenamine derivative, a chemical having more than one methenamine unit, a chemical having multiple R groups conjugated to at least one methenamine unit, a compound having a pharmaceutically acceptable carrier for injection, a compound having a pharmaceutically acceptable carrier for oral ingestion, a compound having a pharmaceutically acceptable carrier appropriate for non-parenteral administration.

18. A method as in claim 16, wherein said providing step comprises providing a methenamine containing compound having a dosage unit appropriate for treating a cancer patient in a treatment protocol.

19. A method to generate an addutive or synergistic anticancer effect by co-administration of the anticancer composition of claim 1 with another anticancer therapy.

20. A method as in the claim 19, the other anticancer therapy comprising a chemical drug.

21-23. (canceled)
